(19) United States
(12) Patent Application Publication
(10) Pub. No.: US 2015/0050372 A1
(43) Pub. Date: Feb. 19, 2015

(54) EXTRACT OF RHUS COPALLINA AS PHARMACEUTICAL

(71) Applicant: Bionorica SE, Neumarkt (DE)

(72) Inventor: Annette Abhau, Syke (DE)

(21) Appl. No.: 14/366,183

(22) PCT Filed: Dec. 19, 2012
(86) PCT No.: PCT/EP2012/076226

§ 371 Date: Nov. 3, 2014

(30) Foreign Application Priority Data
Dec. 19, 2011 (EP) 11194249.6

(51) Int. Cl. A61K 36/22; A23L 1/30; A23L 2/02

(52) U.S. Cl. CPC A61K 36/22; A23L 2/02; A23L 1/3002; A23V 2002/00
USPC 424/774

(57) ABSTRACT

The subject matter of the invention is a pharmaceutical comprising an extract produced by extraction of the leaves of Rhus copallina with a mixture of alcohol and water.

The invention relates to an extract of the leaves of Rhus copallina L. and a pharmaceutical or nutritional supplement that can be obtained therefrom.

There is a large demand for new pharmaceuticals or nutritional supplements for treating gastrointestinal disorders.

The Rhus genus of plants has 150 to 250 species and belongs to the family Anacardiaceae. Several species of the Rhus genus have economic significance for the production of tanning agents and colorants. All species are toxic to a greater or lesser extent. Several Rhus species can cause skin irritation.

U.S. Pat. No. 531,610 discloses the use of water-repellent coating layers and coating varnishes that contain the resin and/or the juice of Rhus copallina.

U.S. Pat. No. 7,528,232 B2 discloses methods and compositions that inhibit pathogen proliferation. In that case, individual proteins extracted from plants are used as antibiotics against human and animal pathogens. According to U.S. Pat. No. 7,528,232, similar endogeneous proteins can be obtained from plants, e.g. from Rhus copallina, by means of a screening method. A specific method for Rhus copallina or a specific protein from Rhus copallina are not disclosed.

U.S. Pat. No. 7,501,557 B1 relates to a system with which proteins and protein products are transported to the surface of plants, and to a method for separating proteins from the plant. U.S. Pat. No. 7,501,557 B1 proposes that Rhus copallina be provided with the genetic construct disclosed in U.S. Pat. No. 7,501,557 B1 for this purpose, for example.

The dissertation authored by Annette Abhau entitled “The survival of a Malagasy lemur species Propithecus verreauxi coquereli in captivity: The vital role of a self-selected plant diet” (released on 20 Jun. 2007; http://deposit.d-nb.de/cgi-bin/dokserv?idn=984680519 discusses, inter alia, the exact qualitative and quantitative analysis of the Rhus copallina plant as an essential nutrient for the aforementioned species of lemur, which is also referred to as Sifaka. In the dissertation, the stemless leaves were used for the analyses and investigations. The nutrient content of the leaves was determined using the Weender method. In order to analyze the ingredients of Rhus copallina, the leaves of Rhus copallina were extracted with a mixture of 80% methanol and 20% water (V/V). The leaves, having been chopped up, were pre-extracted with n-hexane before the preparative isolation of individual compounds with the aid of a polyamide column, followed by preparative HPLC technology.

Isobutyl methyl ketone and water were used for gravimetric isolation.

John K. Crellin and Jane Philpott (A Reference Guide to Medicinal Plants, 1989 Duke University Press, 2^{nd} Edition 1997, pages 417-419) provide an overview of the use of various Rhus species as household remedies.

The following are described for R. glabra L. and R. copallina

a) the use as a medicinal tea that is produced by cooking the berries in water, as cough syrup, for gargling, and for the kidneys;

b) the external use of the leaves for swelling and ulcers, and as fragrances and flavorings for tobacco and as dye, and

c) the use of the berries (ground up in a mixer, stained, then mixed with water and sugar) as a soft-drink replacement and for the treatment of colds.

John K. Crellin and Jane Philpott also describe the medicinal use of Rhus coriaria and the related Cotinus coggygria Scop. as binding agents, for dehydration and for the treatment of colds. These applications have been known since 1597. It is also known to use Rhus coriaria for “fluxes of the belly” and diarrhea. The use thereof as an antipyretic, and the cooling and astringent properties of the berries are also mentioned. Other traditional applications of these Rhus species include the astringent properties of the leaves, roots, and berries of R. coriaria, the use thereof in the treatment of sore throat and afflictions of the oral mucosa, and in the treatment of diarrhea, burns, and hemorrhoids in conjunction with R. coriaria, and the use of the leaves and berries of R. glabra in the treatment of gastrointestinal disorders.

Rhus glabra, Rhus typhina and Rhus aromatica are known from traditional North American medicine, namely as astringents, for use in the treatment of digestive disorders, for gargling and as an oral rinse, and for disorders of the skin and bladder. The leaves are not used in this case, but rather only the peel, roots, and fruit (in “Medicinal Plants of the world” by Ben-Erik van Wyk and Michael Wink).

A few species (such as Toxicodendron species) cause skin irritation.

The problem addressed by the present invention is that of providing a new pharmaceutical. The subject matter of the invention, therefore, is a pharmaceutical or nutritional supplement containing aqueous-alcoholic extracts of Rhus copallina L. and the use thereof as a pharmaceutical or a nutritional supplement.

The subject matter of the invention is a pharmaceutical, in particular, comprising an extract produced by extraction of the leaves of Rhus copallina L. with a mixture of alcohol and water.

The subject matter of the invention is a pharmaceutical, in particular, comprising an extract produced by extraction of the leaves of Rhus copallina L. with a mixture of alcohol and water.

The subject matter of the invention is also a pharmaceutical compound comprising an extract produced by extraction of the leaves of Rhus copallina L. with a mixture of alcohol and water and, optionally, further auxiliary agents and additives.

An alcohol-water extract from leaves of Rhus copallina has not yet been used or described for use as a pharmaceutical or a nutritional supplement.

In preferred embodiments of the invention, the extract is produced by extracting the leaves of Rhus copallina L. with ethanol and water. In another preferred embodiment of the invention, the extract is produced by extracting the leaves of Rhus copallina L. with methanol and water. The extraction can be performed with a mixture, for example, that comprises at least 20% or 30% ethanol/methanol, preferably 40% or 50% ethanol/methanol, particularly preferably 60%, 70% or 80% ethanol or methanol. A particularly preferred embodiment of the invention relates to a pharmaceutical or a nutritional supplement, wherein the extraction is performed with a mixture of alcohol and water, for example a mixture of at least 85% ethanol or methanol, preferably 90% or 95% ethanol or methanol or more, up to the use of pure ethanol or methanol for the extraction. In a particular embodiment of the invention, the extracting agent can contain ethanol and water as well as up to 10% of other solvents, such as ethyl acetate, for example. Ethanol is preferred according to the invention.

The pharmaceutical or nutritional supplement is suitable for animals, in particular livestock, and preferably humans. A preferred embodiment of the invention relates to the pharmaceutical or nutritional supplement for the (specific) use on humans. A further particular embodiment relates to the pharmaceutical for the (specific) use on humans who suffer from chronic stomach and/or intestinal disturbances.

The invention therefore relates to the use of the pharmaceutical for the treatment of gastrointestinal disorders, in particular chronic stomach and/or intestinal disturbances, comprising the regulation of the gastrointestinal tract, for general digestive problems, such as bloating, colic, and/or constipation, for stomach problems, such as a sensation of fullness and discomfort, stomach and/or abdominal pain.

A further particular embodiment of the invention relates to the specific application for children past the stage of early infancy. A further particular embodiment of the invention relates to the use in humans who are immunodeficient, who suffer from intestine bacterial overgrowth or have an illness associated with increased gastrointestinal permeability, such as inflammatory intestinal diseases such as Crohn's disease, colitis ulcerosa, diabetes, asthma, allergic skin diseases, atopic dermatitis (neurodermitis).

Particularly preferred embodiments of the invention relate to pharmaceuticals for the (specific) use as an antidiarrheal agent, an antiphlogistic agent, an analgesic, an antibiotic, an antimycotic, an agent for treating the skin and mucous membranes, including regeneration, diarrhea, inflammation, wound infections, gastrointestinal disorders, infections, sepsis, or as an astringent, a hemostyptic agent, an agent for treating itching, bleeding, and for treating atopic dermatitis (neurodermitis), and spasms (spasmolytic).

Surprisingly, it was found that an extract, in particular an aqueous-alcoholic extract from the leaves of Rhus copallina has antiphlogistic, antiinflammatory, antipruritic, astringent, antiseptic, and antibacterial properties.

A further subject matter of the invention is, in particular, the (specific) use of the pharmaceutical or nutritional supplement for the treatment of gastrointestinal disorders.

The invention relates to the use of the pharmaceutical or nutritional supplement for the treatment and prevention of gastrointestinal disorders, including chronic-inflammatory intestinal diseases such as Crohn's disease, colitis ulcerosa, diarrhea, gastroenteritis, hemorrhoidal complaints, irritable colon, a bacterial overgrowth with resistant and/or non-obligatorily und/oder obligatorily pathogenic organisms. The invention also relates to the use of the pharmaceutical or nutritional supplement for toxin binding, in particular for exotoxins such as the cholera toxin originating from Vibrio cholerae, for example, and for other toxin-producing, enteropathogenic bacteria, such as EHEC, for example.

The invention relates to the use of the pharmaceutical or the nutritional supplement for reducing increased gastrointestinal permeability.

The (specific) applications according to the invention comprise the use of the pharmaceutical for the treatment and/or prevention and/or aftercare of the aforementioned indications.

The invention also relates to the (specific) application of the pharmaceutical or nutritional supplement for cancer and precancerous stages in the gastrointestinal tract, such as intestinal cancer or stomach cancer, and for organisms having carcinogenic potential, such as Helicobacter pylori.

A further subject matter of the invention therefore relates to eradication therapy using the pharmaceutical according to the invention.

The invention relates to the (specific) application against certain problem bacteria such as methicillin-resistant Staphylococcus aureus and other strains of Staph. aureus, and Pseudomonas aeruginosa, which can become a vital problem for patients who are immunosuppressed or hospitalized.

The invention relates to the (specific) application against organisms that occur on the skin and/or mucous membrane, such as Corynebacterium acnes.

The definitions and terms “stomach and/or intestinal disturbances, chronic-inflammatory intestinal diseases, diarrhea, gastroenteritis, hemorrhoidal complaints, irritable colon, Crohn's disease, cholera, digestive problems, such as bloating, colic, and/or constipation, stomach problems, such as a sensation of fullness and discomfort, stomach and/or abdominal pain” are found in the Klinischen Wörterbuch Pschyrembel, 2012 Edition, De Gruyter Verlag, Berlin, for example.

The administration of the pharmaceutical can be oral, rectal, dermal, or mucosal. A particularly preferred embodiment of the invention relates to the oral, dermal, or mucosal administration or application of the pharmaceutical.

The subject matter of the invention also relates to a method for producing an extract of Rhus copallina, wherein the leaves of Rhus copallina are harvested, cleaned, and chopped up, and the leaves are then extracted with a mixture of water and alcohol, such as ethanol or water, for example with a mixture of at least 40% alcohol, preferably 50% alcohol or more, particularly preferably 60% or 80% alcohol or more. In a particular embodiment of the invention, the extracting agent can contain ethanol and water as well as up to 10% of other solvents.

The extraction with alcohol and water is particularly advantageous, because this enables only those ingredients—from the plurality of ingredients in the leaves of Rhus copallina—that have particularly advantageous pharmacological properties to be extracted and concentrated. An overview of the plurality of chemical bonds in Rhus copallina and the content of these substances in the plant are found in the aforementioned dissertation authored by Annette Abhau. From this plurality of ingredients contained in the leaves, only certain ingredients are extracted in certain quantities by means of the extraction method according to the invention. This extraction therefore results in a composition of the extract that has the advantageous pharmacological properties, in particular the antiphlogistic, anti-inflammatory, antipruritic, astringent, antiseptic, and antibacterial properties and the advantageous gastrointestinal effects. The latter includes the intestinal wall sealing effect, the reduction of increased gastrointestinal permeability, slowing of the passage through the intestine, an increase in stool consistency, the reduction of gastrointestinal hypersecretion, and toxin-binding properties against enteropathogenic organisms.

The leaves of Rhus copallina are harvested in the spring or summer or fall. It is also possible to use a mixture of leaves that were harvested in different seasons. For example, leaves can be used that are harvested in May, June and/or October. The leaves can be used fresh, or they can be preserved by drying before extraction. Preferably only the stemless leaves without stalks or branches are used.

The dried or fresh leaves are chopped up (cut) and are optionally pulverized and sifted. The chopped-up leaves are combined with the extracting agent. The extraction is carried out for a certain period of time and under certain conditions. By means of the extraction, the desired ingredients are removed from the leaves of Rhus copallina and concentrated, whereas unwanted companion substances remain in the leaves. Finally, the residue is separated from the leaves and the Rhus copallina extract remains.

The subject matter of the invention is also an extract of Rhus copallina that is produced using this method.

The extraction can take place by means of maceration or percolation, for example. In maceration, the leaves are left in the extracting agent for a defined period of time until a state of equilibrium is reached (not an exhaustive extraction). In percolation, the extracting agent is continuously added to the leaves and the resultant extract is removed.

One embodiment of the invention relates to Rhus copallina liquid extract, wherein the extraction is carried out for up to 7 days, preferably 1 to 5 or 6 days, particularly preferably 2, 3 or 4 days, or, optionally, other known maceration methods.

A short and rapid extraction is particularly preferred, since this prevents the tannins contained in the leaves from decomposing into gallic acid. A rapid extraction can be carried out, for example, within 10 minutes or up to 5 hours, preferably within a time of 30 minutes and 3 hours, particularly preferably approximately 1 hour. Particularly suitable reaction conditions for short and rapid extraction methods are known to a person skilled in the art.

A preferred embodiment of the invention relates to the extraction of the leaves at a relatively high temperature, for example at 30° C., 35° C., 40° C. or 45° C. This enables the extraction time to be shortened. For example, the extraction is carried out at a relatively high temperature and with return flow for 2 or 3 hours.

After the extraction, the residue (the leaves) is separated out and the extract is optionally evaporated to low bulk or is concentrated. The concentration can take place by means of gentle rotary evaporation at 30° C. to 40° C. By means of rotary evaporation, the extract can be evaporated nearly to dryness. The volume of the extract can be reduced by one-third or one-half, for example, or even further.

The drug extract ratio is declared for an extract in order to clarify the ratio between the quantity of the leaves (drug) that are used and the quantity of the extract. The drug extract ratio (DER) specifies the initial amount of drug (drug weight, dry mass) used for the preparation of a certain amount of extract. The DER can always be used when dosing to convert back to the amount of drug used and to compare the quality of various extracts of drugs and draw conclusions about the concentration of the ingredients.

Dried extracts for tablets, for example, or liquid extracts can be produced from the extract of Rhus. Liquid extracts of Rhus copallina have a ratio of 1:1, for example, of extractive substances of one part drug to the amount of liquid extract. Liquid extracts are liquid drug preparations, in the case of which the least amount of extraction fluid possible is used to extract the drug.

In the case of the extract of the leaves of Rhus copallina that is used, a drug/extracting agent ratio of 20 mg drug to 1 ml solvent or extracting agent, or 15 g drug in 150 ml extracting agent is used, for example. The leaves contained up to 28% tannins (m/m), depending on the season. The DER can therefore be varied accordingly. The DER can be 3:1 to 5:1, for example, and other DER are also included according to the invention.

A further subject matter of the invention is also a pharmaceutical composition, preferably in the form of an ointment, a juice, a lotion, a solution, or a suppository, which comprises an aqueous/alcoholic extract of the leaves of Rhus copallina. The ointment, lotion, solution, juice or the suppository can comprise the aqueous/alcoholic extract and the usual carriers, auxiliary agents and additives.

The pharmaceutical compositions or preparations according to the invention can be produced in the form of dosage units. This means that the compositions are present in the form of individual parts, preferably capsules, coated tablets, and ampules, and have an active agent content of the extract according to the invention that corresponds to a fraction or a multiple of an individual dose. The dosage units can contain, for example, 1, 2, 3 or 4 individual doses or ½, ⅓ or ¼ of a single dose. A single dose preferably contains the amount of extract (active agent) according to the invention that is administered in an application and that typically corresponds to a full daily dose, half of a daily dose, one-third of a daily dose, or one-fourth of a daily dose.

In a further preferred embodiment, the galenical formulation of a calcium-coated tablet can be selected, as disclosed in EP1392337.

Non-toxic, inert, pharmaceutically suitable carriers are intended to mean solid, semi-solid or fluid diluents, fillers, and formulation aids of any type, such as a) fillers and extenders, e.g. starches, lactic acid, cane sugar, glucose, mannitol, and silicic acid, b) binders, e.g. carboxymethyl cellulose, alginate, gelatin, polyvinylpyrrolidone, c) moisturizers, e.g. glycerol, d) an agent promoting breakdown, e.g. agar-agar, calcium carbonate and sodium carbonate, e) a dissolution inhibitor, e.g. paraffin and f) resorption accelerators, e.g. quaternary ammonia compounds, g) wetting agents, e.g. cetyl alcohol, glycerol monostearate, h) adsorbents, e.g. kaolin and bentonite and i) lubricants, e.g. talcum, calcium- and magnesium stearate and solid polyethylene glycols or mixtures of the substances mentioned under a) to i).

The tablets, coated tablets, capsules, pills, and granular powder can be provided with the usual coatings and casings, which may optionally contain opacification means, and can also be composed such that these deliver the active agent or agents only or preferably in a certain part of the intestinal tract, optionally in a delayed manner, wherein polymeric substances and wax, for example, can be used as embedding material.

The nutritional supplement according to the invention can also be administered in the form of a composition, as described above. In a further embodiment, the nutritional supplement can be added to any type of food supplement or food, including water.

Food supplements and food within the meaning of this invention are those foodstuffs, in particular, that are defined in the EC Directive 2002/46/EC dated 10 Jun. 2002, although are not limited thereto.

1-13. (canceled)

14. A pharmaceutical comprising an aqueous-alcoholic extract of leaves of Rhus copallina L.

15. The pharmaceutical according to claim 14, wherein the alcohol is ethanol or methanol.

16. The pharmaceutical according to claim 14, wherein the pharmaceutical is utilized to treat humans.

17. The pharmaceutical according to claim 14, in particular an antidiarrheal agent, an antiphlogistic agent, an antiseptic, or an antibiotic, for use to treat humans.

18. The pharmaceutical according to claim 14, wherein the pharmaceutical is utilized to treat gastrointestinal bacterial overgrowth and/or to combat problem bacteria in the region of the skin and mucous membrane, to treat skin and mucous membrane, itching, bleeding, atopic dermatitis (neurodermitis), spasms, diarrhea, inflammation, wound infections, gastrointestinal disorders, infections.

19. The pharmaceutical according to claim 14, wherein the pharmaceutical is utilized to treat or prevent gastrointestinal disorders.

20. The pharmaceutical according to claim 14, wherein the gastrointestinal disorder is selected from the group consisting of
a.) chronic stomach and/or intestinal disturbances, such as the regulation of the gastrointestinal tract, general digestive problems, bloating, colic and/or constipation, stomach problems, such as a sensation of fullness and discomfort, stomach and/or abdominal pain,
b.) chronic-inflammatory intestinal disorders such as Crohn's disease or colitis ulcerosa, diarrhea, gastroenteritis, hemorrhoidal complaints, irritable colon, binding or neutralization of toxins of enteropathogenic organisms, in particular cholera toxin and related toxins, to reduce increased gastrointestinal permeability and disorders associated therewith,
c.) cancer and precancerous stages in the gastrointestinal tract, such as intestinal cancer or stomach cancer,
d.) for the eradication therapy of Helicobacter pylori.

21. The pharmaceutical according to claim 14 for oral, dermal and/or mucosal administration.

22. An ointment, juice, lotion, or suppository comprising an aqueous-alcoholic extract of the leaves of Rhus copallina L. and common auxiliary agents and additives.

23. A method for the production of an extract of Rhus copallina, wherein the leaves of Rhus copallina L. are harvested, cleaned, and chopped up, and the leaves are then extracted with a mixture of water and alcohol, preferably ethanol, for example a mixture of at least 40% alcohol, preferably 50% alcohol or more, particularly preferably 60% to 80% alcohol or more.

24. An extract of Rhus copallina L. that is produced or obtained by means of a method according to claim 23.

25. A pharmaceutical composition comprising an aqueous-alcoholic extract of the leaves of Rhus copallina L., and optionally further auxiliary agents and additives.

26. A nutritional supplement comprising an aqueous-alcoholic extract of the leaves of Rhus copallina L., and optionally further auxiliary agents and additives.

27. The ointment, juice, lotion, or suppository of claim 22, further comprising tannin.
